(19) United States
(12) Patent Application Publication
(10) Pub. No.: US 2023/0138882 A1
(43) Pub. Date: May 4, 2023

(54) INTEGRATIVE WEARABLE HEALTH MONITORING

(71) Applicant: West Affum Holdings Corp., Grand Cayman (KY)

(72) Inventors: Brian D. Webster, Mercer Island, WA (US); Traci S. Umberger, Kirkland, WA (US)

(21) Appl. No.: 17/514,311

(22) Filed: Oct. 29, 2021

(51) Int. Cl. A61B 5/00; A61B 5/339; A61B 5/0205; A61B 5/024; A61B 5/11; A61N 1/39

(52) U.S. Cl. CPC A61B 5/6805; A61B 5/339; A61B 5/0205; A61B 5/02438; A61B 5/11; A61B 5/0006; A61N 1/3904; A61B 2560/0443

(57) ABSTRACT

An integrative wearable health monitoring capable of displaying information about devices and sensors worn using a wearable support structure and according to regulatory compliance rules.

### BACKGROUND

Wearable devices and/or sensors can be used to aid with health monitoring over an extended period and with decision making. Health monitoring devices can be subject to regulatory controls. In the USA, for example, the Food and Drug Administration (FDA) is the regulatory agency with controls over medical devices. The FDA categorizes medical devices as Class I, Class II, or Class III medical devices. This three-tier classification is based on a device's intended use, indications for use, and the risk that it poses. Intended use describes the general purpose or function of the medical device. Indications for use describe the disease or condition the medical device helps diagnose, treat, prevent, or cure.

For heightened cardiac risk individuals, monitoring devices, such as wearable external monitors or wearable defibrillators can be prescribed. Defibrillators are typically classified by the FDA as Class III medical devices and subject to some of the most stringent regulatory compliance rules or controls. Other medical devices can be a Class II, and some can be Class I medical devices. Typically, each type of a regulated device operates within a closed architecture proprietary monitoring, reporting, display system.

Some people who are provided with a Class III regulated device and/or system could also benefit from other Class III, Class II, or Class I regulated devices, and/or even a non-regulated consumer wearable health monitoring device. For example, a patient may suffer from a cardiac condition and can benefit from a Wearable Cardioverter Defibrillator (WCD). A patient may also be diabetic in need of a glucose monitor. The same patient could also benefit from a wearable blood pressure or SpO2 or sleep apnea device, and so on. Additionally, the patient can benefit from wearable health monitor that does not require a regulatory approval, for example activity sensors, step counter, motion sensors sleep sensors, temperature sensors, environmental sensors, etc.

Currently, such devices or sensors, whether regulated or not, provide information using their dedicated platform, which may be separate from any other platform used by another device or sensor. A physician overseeing a patient may be able to view information obtained by a prescribed Class II medical device, but may not be able to use the same platform to view information obtained by another Class II or Class I medical device, or even a non-regulated wearable device using a singular system/display device.

### SUMMARY

This summary is provided to introduce a selection of concepts in a simplified form that are further described below in the Description. This summary is not intended to identify key features of the claimed subject matter, nor is it intended to be used as an aid in determining the scope of the claimed subject matter.

The present disclosure describes examples of a wearable health monitoring device and/or sensor integrative system, which can be configured for adding, removing, replacing devices or sensors. In some embodiments, the system can be configured for discerning whether a device is a regulated device and whether interoperability between a wearer's devices can be established. Such system provides an application configured to confirm the regulatory status of each device joining in and a display configured to display information each device or sensor collect about the wearer or wearer's environment.

In one example of an embodiment, a method for displaying information according to regulatory compliance is described. The method includes providing a health monitoring system configured to display information received from a medical device or a sensor, or both, receiving by a remote computing device information from the medical device, which is subject to a regulatory compliance rules, receiving by the remote computing device information from another device or sensor which is not subject to a regulatory rule, and causing displaying the information from the medical device according to the regulatory compliance rule and according to interoperability of the medical device with the health monitoring system, and displaying the information from the sensor according to interoperability of the sensor with the health monitoring system.

In one example of an embodiment, a method includes connecting a non-medical sensor to the system. The sensor is configured to sense and collect motion information indicative of wearer's activity level, for example, or environmental motion such as detection of a bystander, distance to a bystander, etc. The method also includes monitoring of the cardiac health of the patient for a predetermined period using a wearable defibrillator. While the motion sensor may not be subject to regulatory compliance, the wearable defibrillator is a category III medical device, per FDA classification and subject to regulatory rules and approval process. An application user interface can enable a display of the motion sensor's information and the wearable defibrillator's data.

In some embodiments, the wearable health monitoring system can include a regulatory agency approved wearable medical device, which is a wearable monitoring device (WMD) attached to a wearable support structure configured to be worn by a patient. The WMD includes an interface unit with a port and configured with an application user interface (API) which can enable interoperability with other devices or sensors attached to the support structure and/or worn by the patient.

In some embodiments, a remote computing device may be configured to receive monitored parameter data from a WMD and another sensor. The WMD can be a regulated medical device configured to provide information about a monitored physiological parameter of the wearer. Unlike the WMD, the sensor in this example is not a regulated device. The sensor provides monitored parameter data different from the parameter data provided by the WMD. The sensor data can be a parameter of the wearer or the environment of the wearer. An application user interface can be configured to enable a display of both the WMD information and the sensor information while maintaining integrity of each the WMD and the sensor in accordance with regulation rules. A remote computing device can then be configured to display both WMD parameters and the sensor data.

### BRIEF DESCRIPTION OF THE DRAWINGS

FIG. 1 is a diagram of a sample wearable health monitoring system in accordance with exemplary embodiments described herein.

FIG. 2 is a diagram of an example wearable support structure in accordance with exemplary embodiments described herein.

FIG. 3 is a block diagram of sample embodiments of components of a wearable heath monitoring system in accordance with exemplary embodiments described herein.

FIG. 4 is a block diagram of an example wearable monitoring device in accordance with exemplary embodiments described herein.

FIG. 5 is a flow diagram in accordance with exemplary embodiments described herein.

### DESCRIPTION

Each embodiment described in this disclosure is provided merely as an example or illustration and should not be construed as precluding other embodiments. The illustrative examples provided here are not intended to be exhaustive or to limit the disclosure to the precise forms disclosed.

In the following description, specific details are set forth to provide a thorough understanding of exemplary embodiments of the present disclosure. It will be apparent to one skilled in the art, however, that the embodiments disclosed herein may be practiced without embodying all of the specific details. In some instances, well-known process steps have not been described in detail in order not to unnecessarily obscure various aspects of the present disclosure. Further, it will be appreciated that embodiments of the present disclosure may employ any combination of features described herein.

The present description includes examples of a wearable health monitoring system comprising a medical device (MD), such as a wearable cardioverter defibrillator (WCD), which is configured to collect physiological data about a wearer. The medical device is subject to regulated device rules of a regulatory agency such as the FDA. The wearable health monitoring system may include a device or sensor for collecting parameters different from the physiological data collected by the medical device. Such device or sensor may not be subject to the FDA regulations and as such is a non-medical device (non-MD).

In one embodiment, the wearable health monitoring system includes a support structure, which can be worn by a wearer. The wearer can be a patient. The support structure includes variety of attachment mechanisms allowing to removably attach a device or a sensor to the support structure. The support structure may also be fitted with a communication module, including an application programming interface (API). The system can further include a remote computing device configured to receive information from the medical device or the sensor, or both, via the API of the communication module. Using the API, the system can determine that the medical device and/or the sensor or non-MD is interoperable with the system and display information on the remote computing device in accordance with a regulated or non-regulated device rules.

The present description includes examples of Class III medical devices which can be monitoring devices and treatment devices combined, such as a WCD, and other Class II, Class I, and/or non-medical monitoring devices and/or sensors. Examples of medical monitoring devices include ECG monitors, vital sign monitoring devices, including temperature, blood pressure monitors, heart rate, SPO2, infusion pump devices, etc. In some scenarios, such medical devices can be utilized alone or in combination with other medical devices, or non-medical devices/sensors. Examples of non-medical monitoring devices include motion sensors, carbon monoxide detectors, ambient temperature sensors, ambient pressure or humidity sensors, etc. Monitoring devices, whether medical or non-medical, can be supported by accessories, which can assist with data collection, transfer, trending, and/or alerts. Such accessories can include gateway devices, tablets, mobile devices such as a cell phone or a watch.

In embodiments, a monitoring device can be worn, for example when attached to the support structure. When worn, the monitoring device can be referred to as a wearable monitoring device (WMD). An example of a WMD is a medical device such as a WCD, or a non-medical device, such as a motion sensor. A WMD that is a medical device can aggregate monitored physiological parameter data, such as ECG, heart rate, pulse, etc. The WMD can transmit information to a remote computing device. The information can include information about the device itself such as whether the WMD is a Class III or Class II or Class I regulated medical device or a non-medical device and/or information comprising monitored by the device parameter information.

In some instances, a patient may need monitoring for a heart condition as well as glucose levels, and/or blood pressure trend, but also ambient temperature the wearer of the system is exposed to over a period of time. Here, more than one device can be used over a time period. The present disclosure provides for a method and a system configured to display information comprising physiological heart parameters and/or glucose level and/or pressure parameters, and also ambient temperatures, for example.

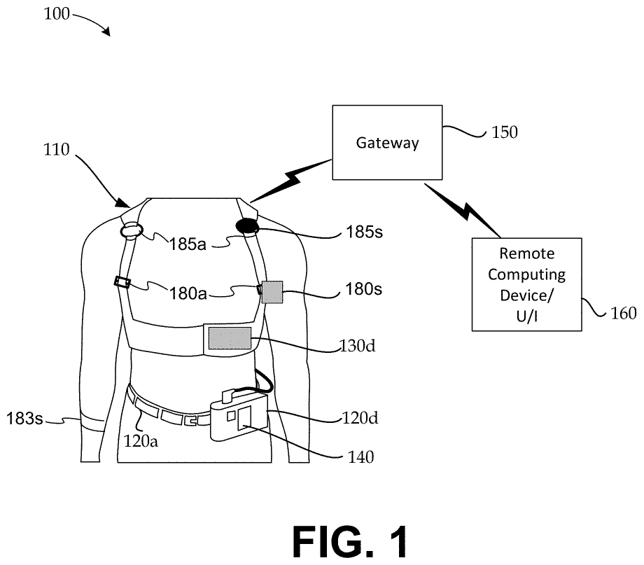

FIG. 1 illustrates an embodiment of an integrative wearable health system 100. The system comprises a wearable support structure 110, also referred to as a scaffold, which can include a garment, a vest, a shirt, a strap, a belt, a band, or the like. In some embodiments, the support structure 110 can be a single component. In other embodiments, the support structure can be modular and/or comprise multiple components or a combination of components such as a vest, a belt, and a wrist or arm attachment, for example.

In one embodiment, as illustrated in FIG. 1 a patient wears a medical device, such as device 120d and/or 130d and/or one or more non-medical device 180s, 185s, 183s. The patient can wear devices over various time periods that may or may not overlap. The medical device 120d, 130d and/or a non-medical device, also referred to here as sensors, 180s, 185s, 183s can transmit information comprising acquired monitored parameters to a remote computing device 160. The remote computing device 160 can include a user interface and display the information. The medical device 120d, 130d and/or the non-medical device 180s, 185s, 183s can be configured to transmit information directly to the remote computing device 160. The medical device and/or the non-medical device can also be configured to transmit information to the remote computing device 160 via a gateway device 150 such as a mobile phone of the wearer, for example.

In one embodiment, a medical device, such as a Class III medical device 120d and/or 130d can be removably attached to the wearable support structure 110 via an attachment mechanism such as attachment mechanism 120a. Sensors 180s, 185s. Devices, meaning medical or non-medical devices, can be added or removed to the wearable support structure 110 using attachment mechanisms such as for example 120a, 180a, 185a. An attachment mechanism can be configured to accommodate a one or more types of devices. In another embodiment, additional device, which can be worn independently of the wearable support structure 110, for example arm-worn device 183s, or an adhesive patch (not shown) can also be integrated when determined interoperable with the system by the API. The location of attachment, for example around the torso versus on the belt can be based on the type of device used and/or the physiological parameter monitoring objective, weight distribution, and/or other considerations. For example, a wearable cardiac defibrillator may need to be positioned around a wearer's torso in proximity to the heart while a step counter can be positioned closer to an extremity. Some sensors or devices can be relocated, for example if the wearer finds it more comfortable to move the sensors 180s, 185s from one side to another of the support structure, s/he can do so by using similar attachment mechanisms elsewhere on the support structure 110.

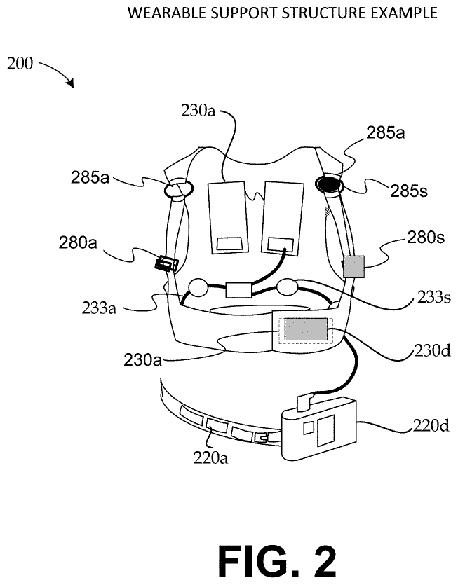

FIG. 2 illustrates an example of a wearable support structure 200. In some embodiments, the support structure 200 can include attachment mechanisms configured for attaching a medical device or a non-medical device/sensor. For example, as illustrated in FIG. 2, the support structure 200 can include an upper, vest or garment-like, support structure, and a lower, belt-like, support structure, each configured with attachment mechanisms for securing wearable medical device components and/or sensor components in locations around a torso or around hips of a wearer. The attachment mechanisms are configured to allow attachment and detachment of devices to the support structure.

In one example, a Class III medical device may have distributable components itself. For example, a WCD may include ECG electrodes and therapy, also referred to as defibrillation, electrodes, and a monitor/therapy unit. Components of WCD can be distributed around the wearable support structure 200 such that the ECG electrodes are distributed around the torso and the defibrillation electrodes are distributed to provide therapy to the heart in positions different from that of the ECG electrodes and the monitor/therapy unit connected to both the ECG and defibrillation electrodes can be attached to the belt. ECG electrodes 233s of the WCD device are shown as connected and attached to the support structure 200 via a garment embedded attachment 233a in the upper support structure. Another component, therapy or defibrillation electrodes, of the WCD can also be secured to the support structure via attachment 230a, which can be located in a form of a pocket for a defibrillation electrode placement in the back of the upper support structure 200. Defibrillation electrode 230d of the WCD can also be placed in a front pocket, similar to that of attachment 230a of the upper support structure as shown in FIG. 2. Another component of the WCD, a monitor 220d which is connected to the ECG and the defibrillation electrodes can be attached to the lower, belt, support structure via an attachment mechanism 220a around the belt. Components of other sensors or devices can also be distributed about the support structure independently of the WCD.

In some embodiments, placement of components of WMDs and sensors can be based on comfort, weight distribution in addition to optimization for physiological data acquisition, and/or personal preference. In some embodiments, a wearer's needs or condition may provide for change in the physiological data and removal or relocation of a WMD or sensor. For example, a sensor 285s or 280s can be attached to the support structure via attachment mechanism, such as attachment mechanism 285a or 280a.

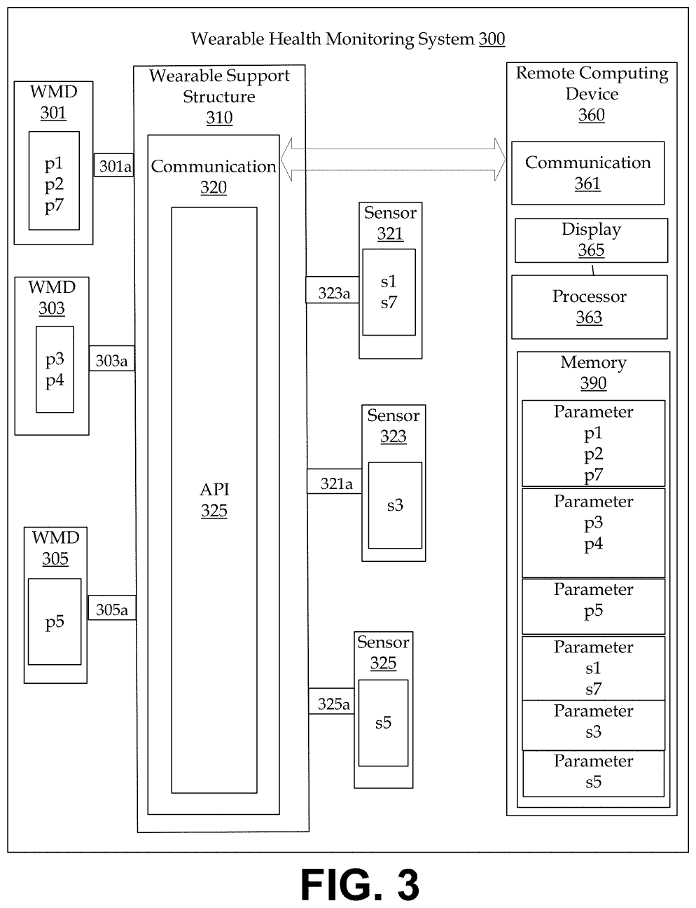

FIG. 3 illustrates an example embodiment of an integrative wearable health monitoring system 300. The integrative wearable health monitoring system 300 includes a wearable support structure 301, which is configured to support at least one medical device, also referred to as a wearable monitoring device (WMD) and/or at least one non-medical device or sensor (sensor). In one embodiment, the wearable support structure 301 comprises an API 325 and a communication module 320.

The integrative health monitoring system 300 can also include a remote computing device 360. The remote computing device 360 comprises a remote device communication module 361, a display 365, a processor 363, a memory 390. The memory 390 is configured to store parameters p1, p2, p7 obtained by the WMD 301, and/or parameters p3, p4 of the WMD 303, and/or parameter p5 obtained by WMD 305. The memory 390 is also configured to store parameters obtained by any of the attached sensors 321, 323, and/or 325 by sensor 321, 323, and/or 325, respectively. The parameters acquired by any one WMD and/or sensor can then be transmitted by the communication module 320 of the wearable support structure 310 to the remote computing device 360 via the remote computing device communication module 361, processed by the processor 363 and displayed on the display 365.

FIG. 3 is a schematic of an integrative health monitoring system 300 in which a remote computing device 360 can access and display information from various independent wearable monitoring devices, such as a WMD 301, 303, 305 and/or sensors 321, 323, 325 via an API 325 of the communication module 320. The remote computing device 360 may receive information from various wearable monitoring devices WMDs 301, 303, 305, and/or various sensors 321, 323, 325, which can be coupled to a wearable support structure 310 via attachment mechanisms 301a, 303a, 305a, 323a, 323a, 325a. The communication module 320 of the wearable support structure 310 is configured to include an API 325. The communication module 320 can be integrated into the wearable support structure 310 or can be a separate device, such as the gateway device 150 shown in FIG. 1. The remote computing device can receive information from a monitoring device or a sensor and determine interoperability of the device or sensor with the wearable health monitoring system. When interoperability is confirmed by the processor 363 of the remote computing device 360, the communication module 361 can cause the display 365 to display parameters acquired by the device or sensor and cause the memory 390 to store the parameters in accordance with any medical device regulations.

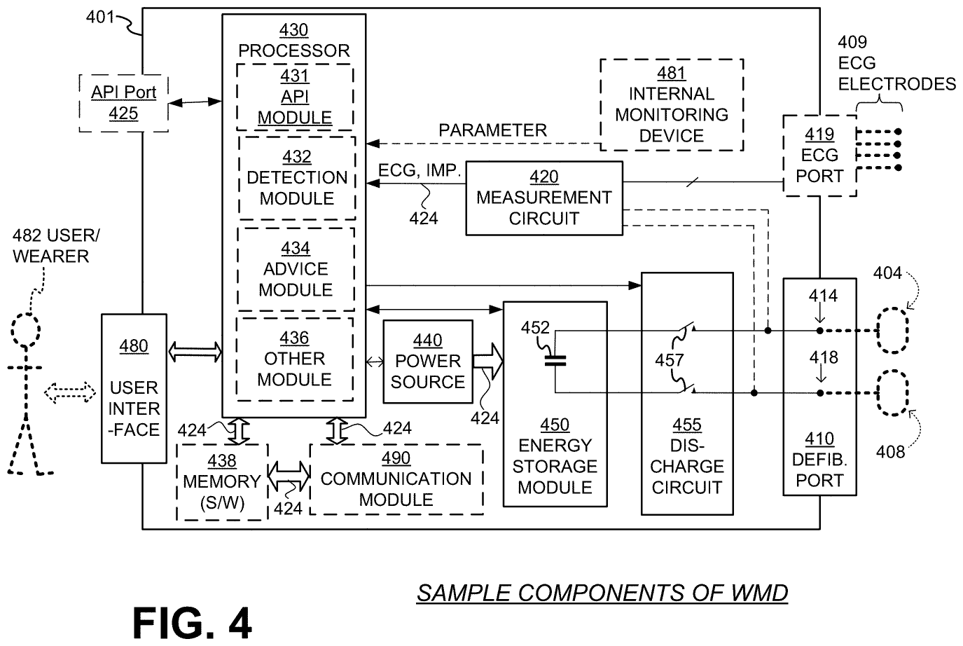

In one example, a wearer may be wearing a wearable support structure 310. One or more devices such as a WMD 301, 303, and/or 305, and/or one or more non-medical devices, such as sensor 321, 323, and/or 325 may be removably attached to the wearable support structure 310 via attachment mechanisms, such as 301a, 303a, 305a, and/or 321a, 323a, 325a. The WMDs may include two or more medical devices with different regulatory classifications. The one or more devices may transmit information to a remote computing device 360 via an API 325 using the communication module of the WMD, such as the communication module 490 illustrated in FIG. 4, the communication module 320 of the support structure, and the communication module 361 of the remote computing device 360.

In embodiments, a WMD 301, 303, or 305 may be a WCD, as described further with reference to FIG. 4. WMD can be a Holter monitor, MCOT, MCT, a glucose monitor, a blood pressure device, a vital sign monitor, an SpO2 device, and the like. In some embodiments, the WMD 301, 303, or 305, and/or the senor 321, 323, 325 may include one or more smart devices such as a smart watch, a mobile phone, a computer, or another device capable of communicating over the Internet.

In some embodiments, a device may transmit information to a remote computing device using an API in a gateway device 150 as shown in FIG. 1. The information may be raw data or may be filtered data collected from the one or more devices and/or sensors. In some embodiments, a remote computing device may request the data transfer from a device connected to the wearable support structure. The periodicity of data transfer may be predetermined intervals or may be adjusted.

The memory 390 of the remote computing device may include random access memory (RAM), read only memory (ROM), flash RAM, and/or other types. The memory 390 may store computer-readable, computer-executable software/firmware code including instructions that, when executed, cause the processor 363 to perform various functions (e.g., determine if a device is interoperable with wearable monitoring health system, and/or determine if the device is a regulated medical device, and/or display information in accordance with the regulated device rules or display information in accordance with non-regulated device rules, etc.). In some embodiments, the processor 363 may include an intelligent hardware device, e.g., a central processing unit (CPU), a microcontroller, an application-specific integrated circuit (ASIC), etc.

In some embodiments, the memory 390 can contain, among other things, the Basic Input-Output system (BIOS) which may control basic hardware and/or software operations such interactions and workings of the various WMDs and/or sensors. For example, the memory 390 may contain various modules to store parameters from various WMDs and/or sensors.

In one embodiment, a wearable support structure 310, shown in FIG. 3, includes a variety of attachment mechanisms 301a, 303a, 305a configured to attach a medical device WMD 301, WMD 303, WMD 305. The wearable support structure 310 can be further configured to include a variety of attachment mechanisms 321a, 323a, 325a configured to attach a non-medical device, such as sensor 321, 323, 325.

In one embodiment, the attached WMD 301 is configured to obtain physiological parameters p1, p2, p7, while WMD 303 is configured to obtain physiological parameters p3 and p4, and WMD 305 is configured to obtain a physiological parameter p5. For example, WMD 301 can be a WMD configured to obtain physiological parameter p1 comprising any of the vital signs, for example temperature, and a physiological parameter p2 comprising a blood sugar level, and/or p3 comprising SpO2 of a wearer. In a further example, WMD 303 can comprise a WCD configured to obtain parameter p3 which can be an ECG signal and/or parameter p4 which can be a heart rate. In still another example, WMD 305 can be a device monitoring any of the wearer's other vital signs not monitored by WMD 301 which could include pulse rate, respiration rate, or blood pressure, as parameter p5.

In one embodiment, sensor 321 can be configured to obtain parameters s1 and s7, for example ambient temperature and pressure, sensor 323 can be configured to obtain parameter s3, for example motion detection, while sensor 325 can be configured to obtain parameter s5, for example Carbon Monoxide presence. Other environmental parameters can include humidity, pollen count, smoke detectors. Parameters obtained from any of the non-medical device/sensor is different from any physiological parameters obtained by any of the WMDs, WMD 301, 303, or 305.

The wearable medical devices WMD 301, 303, 305 may include one or more components. For example, or wearable medical device may include one or more electrodes to detect ECG parameter data, a heart rate parameter data. The wearable medical devices can include a perfusion data, a pulse oximetry, a device for detecting blood flow (e.g., a Doppler device), blood pressure (e.g., a cuff). Medical devices can include an optical detectors, illumination detectors. Devices can include devices configured to sense and detect a change in tissue, motion, heart wall movement, sound, SpO2, and so on.

In some embodiments, the parameter is a trend that can be detected in a monitored physiological parameter of a wearer. A trend can be detected by comparing values of parameters at different times over short and long terms. Parameters whose detected trends can help a cardiac rehabilitation program may include: a) cardiac function (e.g. ejection fraction, stroke volume, cardiac output, etc.); b) heart rate variability at rest or during exercise; c) heart rate profile during exercise and measurement of activity vigor, such as from the profile of an accelerometer signal and informed from adaptive rate pacemaker technology; d) heart rate trending; e) perfusion, such as from SpO2, CO2, or other parameters such as those mentioned above, f) respiratory function, respiratory rate, etc.; g) motion, level of activity and/or bystander detection and/or distance, and so on. Once a trend is detected, it can be stored and/or reported via a communication link, along perhaps with a warning if warranted. The report may aid in monitoring the progress of wearer by correlating information from different devices.

Physiological parameters may be stored remotely and include the wearer's history, event history, and the like. Additional examples of such parameters may include the blood flow, pulsatile change in light transmission or reflection properties of perfused tissue, heart wall motion, breathing sounds, coughing sounds, etc.

In embodiments, the communication module 320 of the wearable support structure 310 can be configured to transmit information from any of the attached devices to a remote computing device 360 using the API 325 and the communication module 320 to communicate with the communication module 361 of the remote computing device 360.

FIG. 4 is a diagram displaying components of an example of a medical device such as a WCD. When attached to a wearable support structure, the WCD is a wearable monitoring device (WMD) as well as a wearable treatment device The components shown in FIG. 4 may be contained within a single housing or may be separated amongst two or more components in communication with each other. A WCD as shown in FIG. 4 may include a communication module 490, processor 430, memory 438, defibrillation port 410, and ECG port 419, among other components. In some embodiments, components are contained within a housing 401 or casing. The housing 401 may comprise a hard shell around the components or may comprise a softer shell for increased wearer's comfort. The housing 401 may be further configured to attach to a support structure via an attachment mechanism, examples of which are illustrated in FIG. 1-3.

The communication module 490, processor 430 including the API module 431, memory 438 (including software/firmware code (SW)), defibrillation port 410, ECG port 419, measurement circuit 420, monitoring device 481, and energy storage module 450 may communicate, directly or indirectly, with one another via one or more buses 424. The one or more buses 424 may allow data communication between the components and/or modules of the WCD. Furthermore, the one or more buses 424 may allow communication between the WCD and the communication module 320 of the wearable support structure 310 and/or the communication module 361 of the remote computing device 360 of FIG. 3.

The memory 438 may include random access memory (RAM), read only memory (ROM), flash RAM, and/or other types. The memory 438 may store computer-readable, computer-executable software/firmware code including instructions that, when executed, cause the processor 430 to perform various functions (e.g., determine shock criteria, determine heart rate, issue shock command, issue alerts, etc.). In some embodiments, the processor 430 may include an intelligent hardware device, e.g., a central processing unit (CPU), a microcontroller, an application-specific integrated circuit (ASIC), etc.

In some embodiments, the memory 438 can contain, among other things, the Basic Input-Output system (BIOS) which may control basic hardware and/or software operations such interactions and workings of the various components of the WCD, and in some embodiments, components external to the WCD. For example, the memory 438 may contain various modules to implement the workings of the WCD and other aspects of the present disclosure.

In some embodiments, the WCD may include a user interface 480 for a user who may be a wearer 482. A user 482 may also be a caregiver, a physician, an EMT, or other authorized user. The user interface 480 may be in addition to or part of the communication module 490. The user interface 480 may display an ECG of the patient, a status of the device, a status of a charge (e.g., a battery charge or an energy storage module), and the like, for example.

In some embodiments, the user interface 480 may include output devices, such as WMD 120d of FIG. 1, which may include visual, audible, or tactile, for communicating to a user by outputting images, sounds or vibrations. Images, sounds, vibrations, and an alert or notification that can be perceived by a person is also called human-perceptible indications (HPIs). Output devices, or HPIs, may include a light(s), a screen to display 140 shown in FIG. 1 what is sensed, detected and/or measured, speakers, and the like. In some embodiments, the screen may provide visual feedback to a third party for their resuscitation attempts and treatment plans. In some embodiments, the speaker may be configured to issue voice prompts, beeps, loud alarm sounds and/or words to warn bystanders, etc.

In some embodiments, the user interface 480 may further include input devices for receiving inputs from users. Such input devices may include various controls, such as pushbuttons, keyboards, touchscreens, one or more microphones, and so on. An input device can be a cancel switch, which is sometimes called an “I am alive” switch or “live man” switch. In some embodiments, actuating the cancel switch can prevent the impending delivery of a shock.

In some embodiments, the WCD may include various components including a defibrillation port 410. The defibrillation port 410 may comprise a socket, opening, or electrical connection in the housing. In some instances, the defibrillation port 410 may include two or more nodes 414, 418. The two or more nodes 414, 418 may accept two or more defibrillation electrodes (e.g., defibrillation electrodes 404, 408). The nodes 414, 418 may provide an electrical connection between the defibrillation electrodes 414, 418 and the WCD. hardwired to the nodes 226, 228.

In some embodiments, components of a WMD, such as a WCD shown in FIG. 4 may include an ECG port 419 in the housing 401. The ECG port 419 may accept one or more ECG electrodes 409 or ECG leads. In some instances, the ECG electrodes 409 sense a patient's ECG signal. For example, the ECG electrodes 409 may record electrical activity generated by heart muscle depolarization, timing, or both. The ECG electrodes 409 may utilize 4-leads to 12-leads or multichannel ECG, or the like. The ECG electrodes 409 may connect with the patient's skin.

In some embodiments, the WMD may include a measurement circuit 420. The measurement circuit 420 may be in communication with the ECG port 419. For example, the measurement circuit 420 may receive physiological parameter signals from ECG port 419. The measurement circuit 420 may additionally or alternatively receive physiological signals via the defibrillation port 410 when defibrillation electrodes 404, 408 are attached to a wearer. The measurement circuit 420 may determine a wearer's ECG signal from a difference in voltage between the defibrillation electrodes 404, 408.

In some embodiments, the WMD may include an internal monitoring device 481 within the housing 401. The monitoring device 481 may monitor at least one additional parameter to the parameters monitored by the WMD. The additional parameter may include physical state of the patient such as temperature, movement, heartrate, pulse, temperature, SpO2 and the like. The additional parameter may also include an environmental parameter, such as ambient temperature, carbon monoxide detection, or the like.

In some embodiments, a WMD may include an internal monitoring device 481 and may be removably attached to a support structure as shown in FIG. 1-3 along with other wearable medical device and/or sensor. With two or more wearable monitoring devices and/or sensors are present, the API 325, shown in FIG. 3, can be configured to provide a unified platform for parameter data from devices and sensors to be transmitted via the communication module 320 to a remote computing device 360, as shown in FIG. 3.

In some embodiments, the WMD may include a power source 440. The power source 440 may comprise a battery or battery pack, which may be rechargeable. In some instances, the power source 440 may comprise a series of different batteries to ensure the WMD has power. For example, the power source 440 may include a series of rechargeable batteries as a prime power source and a series of non-rechargeable batteries as a secondary source. If the wearer is proximate an AC power source, such as when sitting down, sleeping, or the like, the power source 440 may include an AC override wherein the power source 440 draws power from the AC source.

In some embodiments, the WMD such as a WCD may include an energy storage module 450. The energy storage module 450 may store electrical energy in preparation or anticipation of providing a sudden discharge of electrical energy to the patient. In some embodiments, the energy storage module 450 may have its own power source and/or battery pack. In other embodiments, the energy storage module 450 may pull power from the power source 440. In still further embodiments, the energy storage module 450 may include one or more capacitors 234. The one or more capacitors 452 may store an electrical charge, which may be administered to the patient. The processor 430 may be communicatively coupled to the energy storage module 450 to trigger the amount and timing of electrical energy to provide to the defibrillation port 410 and, subsequently, the wearer of the WMD.

In some embodiments, the WMD such as a WCD may include a discharge circuit 455. The discharge circuit 455 may control the energy stored in the energy storage module 450. For example, the discharge circuit 455 may either electrical couple or decouple the energy storage module 450 to the defibrillation port 410. The discharge circuit 455 may be communicatively coupled to the processor 430 to control when the energy storage module 450 and the defibrillation port 410 should or should not be coupled to either administer or prevent a charge from emitting from the WCD. In some embodiments, the discharge circuit 455 may include on or more switches 457. In further embodiments, the one or more switches 457 may include an H-bridge.

In some embodiments, the WMD may include a communication module 2490. The communication module 490 may establish one or more communication links with either local hardware and/or software to the Wearable Support Structure 310 of FIG. 3 and/or the remote computing device 360, shown in FIG. 3. In some embodiments, the communication module 490 may include one or more antennas, processors, and the like. The communication module 490 may communicate wirelessly via radio frequency, electromagnetics, local area networks (LAN), wide area networks (WAN), virtual private networks (VPN), RFID, Bluetooth, cellular networks, and the like. The communication module 216 may facilitate communication of data and commands such as patient data, episode information, therapy attempted, CPR performance, system data, environmental data, and so on. In some embodiments, the communication module 490 may include a display screen to display messages to the patient, similar to the display screen 140 shown in FIG. 1 and/or display screen on a remote computing device 160, such as shown in FIG. 1. In some embodiments, the display screen may be a touch screen, backlit screen, passive, reflective LCD screen or the like.

In further embodiments, the communication module 360 of FIG. 3 and/or the communication module 490 of FIG. 4 may include one or more LEDs which may also be used to convey information to the patient. In some embodiments, the LED brightness may be modulated, the LEDs may be color changing, and the like. In some embodiments, if multiple LEDs are present, each LED may represent various bits of information. For example, one LED may represent heartrate information and enable the patient to quickly determine their heart is operating normally. Another LED may represent the heartrate signal to ensure the patient the heartrate readings are being properly transmitted. Another LED may also represent system status and allow the patient to easily ascertain that the system is fully functioning.

In some embodiments, the processor 430 may execute one or more modules. For example, the processor 430 may execute a detection module 432 and/or an advice module 434 and/or API module 431, and/or other module 436. The detection module 432 may be a logic device or algorithm to determine if any or a variety of thresholds are exceeded which may require action of the WMD. For example, the detection module 432 may receive and interpret all of the signals from the ECG port 419, and/or if the WMD is a WCD, the defibrillation port 410, the internal monitoring device 481, an API Port 425 configured to connect with the API 325 shown in FIG. 3, and the like. The detection module 432 may process the parameter information to determine the wearer is still conscious and/or the wearer's status. If any parameter indicates the wearer may be experiencing a change in status or abnormal parameters episode, such as parameters out of predetermined normal ranges for the wearer, the detection module 432 may activate the advice module 434.

The advice module 434 may receive data from the detection module 432 and perform a series of actions. For example, an episode may merely be a loss of battery power at the power source 440 or the energy storage module 450, or one or more electrodes (e.g., ECG electrodes, defibrillation electrodes) may have lost connection. In such instances, the advice module 432 may trigger an alert to the wearer and/or to the remote computing device. This may include activating the other module 436. If an episode is a health risk, such as a cardiac event, the advice module 434 may begin a series of steps. This may include issuing a warning to the wearer, issuing a warning to a third party at a remote computing device, priming the energy storage module 450 for defibrillation, and the like.

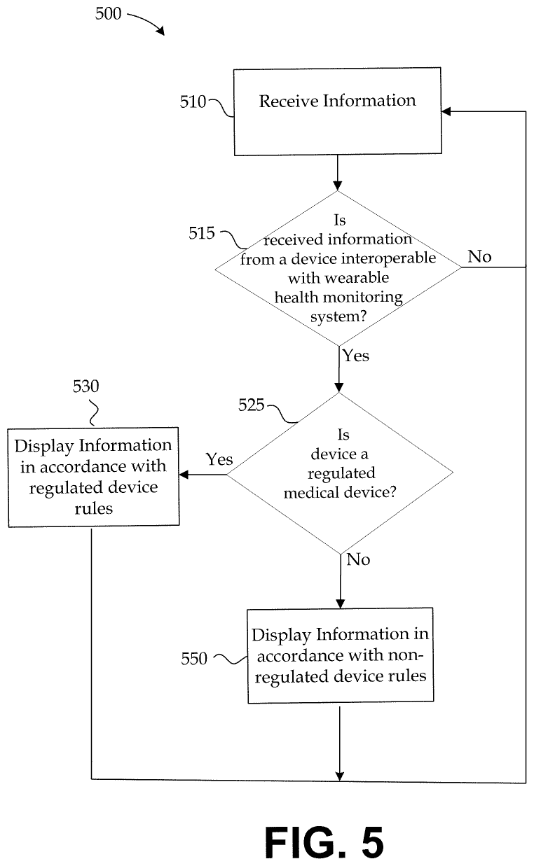

FIG. 5 is a flow chart diagram illustrating an example method 500 for an integrative wearable health system, in accordance with various aspects of the present disclosure. The example method 500 is described with reference to aspects of one or more of the system, components, etc., described herein.

At block 510, the method 500 includes receiving information from at least one WMD and/or sensor as described with reference to FIGS. 1-4. For example, a remote computing device 160, 360 as described with reference to FIGS. 1 and 3, may receive information indicating a device is attached to a support structure. The remote computing device may then determine if the attached device is interoperable with the wearable health monitoring system using API and if the device is interoperable, the remote device may then determine if it is a medical or non-medical device. If the device is a medical device, the remote device may then display information in accordance with the regulatory rules the device is subject to. If the device is a non-regulated device, also referred to here as a non-medical device/sensor, then the remote computing device can display information in accordance to non-medical device rules.

At step 515, the method determines whether a device from which information is received by the remote computing device 160, 360 as shown in FIGS. 1 and 3, is interoperable with the integrative health monitoring system. In one embodiment, the remote computing device receives information initially when a device is attached to the wearable support structure as shown in FIGS. 1-3. If the device transmitting information is interoperable with the integrative wearable health monitoring system, at step 525 the method determines whether the device is a regulated device. If the device is a regulated device, information received can be displayed at block 530 in accordance with the regulated device rules. On the other hand, if the device or sensor is not a regulated device or sensor, at block 550, information received can be displayed in accordance with non-regulated device rules.

A person skilled in the art will be able to practice the present invention after careful review of this description, which is to be taken as a whole. Details have been included to provide a thorough understanding. In other instances, well-known aspects have not been described, in order to not obscure unnecessarily this description.

Some technologies or techniques described in this document may be known. Even then, however, it is not known to apply such technologies or techniques as described in this document, or for the purposes described in this document.

This description includes one or more examples, but this fact does not limit how the invention may be practiced. Indeed, examples, instances, versions or embodiments of the invention may be practiced according to what is described, or yet differently, and also in conjunction with other present or future technologies. Other such embodiments include combinations and sub-combinations of features described herein, including for example, embodiments that are equivalent to the following: providing or applying a feature in a different order than in a described embodiment; extracting an individual feature from one embodiment and inserting such feature into another embodiment; removing one or more features from an embodiment; or both removing a feature from an embodiment and adding a feature extracted from another embodiment, while providing the features incorporated in such combinations and sub-combinations.

In general, the present disclosure reflects preferred embodiments of the invention. The attentive reader will note, however, that some aspects of the disclosed embodiments extend beyond the scope of the claims. To the respect that the disclosed embodiments indeed extend beyond the scope of the claims, the disclosed embodiments are to be considered supplementary background information and do not constitute definitions of the claimed invention.

In this document, the phrases “constructed to”, “adapted to” and/or “configured to” denote one or more actual states of construction, adaptation and/or configuration that is fundamentally tied to physical characteristics of the element or feature preceding these phrases and, as such, reach well beyond merely describing an intended use. Any such elements or features can be implemented in a number of ways, as will be apparent to a person skilled in the art after reviewing the present disclosure, beyond any examples shown in this document.

Incorporation by reference: References and citations to other documents, such as patents, patent applications, patent publications, journals, books, papers, web contents, have been made throughout this disclosure. All such documents are hereby incorporated herein by reference in their entirety for all purposes.

Parent patent applications: Any and all parent, grandparent, great-grandparent, etc. patent applications, whether mentioned in this document or in an Application Data Sheet (“ADS”) of this patent application, are hereby incorporated by reference herein as originally disclosed, including any priority claims made in those applications and any material incorporated by reference, to the extent such subject matter is not inconsistent herewith.

Reference numerals: In this description a single reference numeral may be used consistently to denote a single item, aspect, component, or process. Moreover, a further effort may have been made in the preparation of this description to use similar though not identical reference numerals to denote other versions or embodiments of an item, aspect, component or process that are identical or at least similar or related. Where made, such a further effort was not required, but was nevertheless made gratuitously so as to accelerate comprehension by the reader. Even where made in this document, such a further effort might not have been made completely consistently for all of the versions or embodiments that are made possible by this description. Accordingly, the description controls in defining an item, aspect, component or process, rather than its reference numeral. Any similarity in reference numerals may be used to infer a similarity in the text, but not to confuse aspects where the text or other context indicates otherwise.

The claims of this document define certain combinations and subcombinations of elements, features and acts or operations, which are regarded as novel and non-obvious. The claims also include elements, features and acts or operations that are equivalent to what is explicitly mentioned. Additional claims for other such combinations and subcombinations may be presented in this or a related document. These claims are intended to encompass within their scope all changes and modifications that are within the true spirit and scope of the subject matter described herein. The terms used herein, including in the claims, are generally intended as “open” terms. For example, the term “including” should be interpreted as “including but not limited to,” the term “having” should be interpreted as “having at least,” etc. If a specific number is ascribed to a claim recitation, this number is a minimum but not a maximum unless stated otherwise. For example, where a claim recites “a” component or “an” item, it means that the claim can have one or more of this component or this item.

In construing the claims of this document, the inventor(s) invoke 35 U.S.C. § 112(f) only when the words “means for” or “steps for” are expressly used in the claims. Accordingly, if these words are not used in a claim, then that claim is not intended to be construed by the inventor(s) in accordance with 35 U.S.C. § 112(f).

What is claimed is:

1. A method for displaying information according to regulatory compliance rules, the method comprising:
receiving by a remote computing device, information from a first device removably attached to a wearable support structure of a wearable health monitoring system;
determining, by the remote computing device, from the received information that the first device is interoperable with the wearable health monitoring system,
determining, by the remote computing device, from the received information that the first device is a regulated medical device or a non-regulated device;
responsive to a determination that the first device is a regulated device, displaying, by the remote computing device, the received information in accordance with a regulated device compliance rule; and
responsive to a determination that the first device is a non-regulated device, displaying, by the remote computing device, the received information in accordance with a non-regulated device rule.

2. The method of claim 1, wherein the information received by the processor from the first device includes information about classification of the first device in Class III, Class II, or Class I of FDA-regulated medical device.

3. The method of claim 1, wherein the information received further comprises parameter data monitored by the first device.

4. The method of claim 3, wherein the information received from the first device determined as being a regulated device further includes physiological parameters of a wearer wearing the first device attached to the support structure.

5. The method of claim 4, wherein the physiological parameters include an ECG signal, a heart rate, and/or any vital sign parameter.

6. The method of claim 1, wherein the information from the first device not being a regulated device includes motion information.

7. The method of claim 1 further comprising;
receiving second information from a second device removably attached to the wearable support structure of a wearable health monitoring system;
determining, by the remote computing device, from the received second information that the second device is interoperable with the wearable health monitoring system;
determining, by the remote computing device, from the received second information that the first device is a regulated medical device or a non-regulated device;
responsive to a determination that the second device is a regulated device, displaying, by the remote computing device, the received second information in accordance with a regulated device compliance rule; and
responsive to a determination that the second device is a non-regulated device, displaying, by the remote computing device, the received second information in accordance with a non-regulated device rule.

8. The method of claim 1 wherein the determining that the first device is interoperable with the wearable health monitoring system includes an application programming interface (API).

9. The method of claim 7 wherein the first device is a medical device and the second device is a sensor.

10. A wearable health monitoring system, comprising:
a first regulatory agency approved medical device (first MD);
an interface unit coupled to the first MD, the first interface comprising a port and configured to interoperate with third party devices using an application programming interface (API);
a support structure configured to be worn by a wearer and to removably attach:
a sensor, the sensor distinct from the first MD and configured to provide information of a physiological parameter of the wearer sensed by the sensor to the port in accordance with the API, and
a second regulatory agency approved medical device (second MD) configured to provide information associated with the wearer generated by the second MD to the port in accordance with the API.

11. The wearable health monitoring system of claim 10, wherein the port comprises a wireless communication port.

12. The wearable health monitoring system of claim 10, further comprising a remote computing device coupled to receive, from the interface unit, information comprising at least part of the information provided by the sensor, information associated with the wearer generated by the first MD, and information generated by the second MD.

13. The wearable health monitoring system of claim 12, wherein the remote computing is configured to generate a report using information received from the interface unit.

14. The wearable health monitoring system of claim 10, wherein the port comprises a wireless communication port.

15. A wearable health monitoring system comprising:
a first device subject to first regulatory rule and configured to monitor a first parameter;
a second device not subject to the first regulatory rule and configured to monitor a second parameter different from the first parameter monitored by the first device;
a support structure configured to be worn by a wearer, the support structure comprising:
a first attachment mechanism configured to removably attach the first device to the wearable support structure; and
a second attachment mechanism configured to removably attach the second device to the wearable support structure; and
a processor configured to:
determine that the first device is interoperable with the wearable health monitoring system,
based on a determination that the first device is interoperable with the wearable health monitoring system cause a display to display information comprising the first parameter in accordance with the first regulatory rule, and based on a determination that the first device is not interoperable with the wearable health monitoring system, not cause the display to information comprising the first parameter,
determine that the second device is interoperable with the wearable health monitoring system, and
based on a determination that the second device is interoperable with the wearable health monitoring system, cause the display to display information comprising the second parameter in accordance to not being subject to the first regulatory rule and based on a determination that the second device is not interoperable with the wearable health monitoring system, not cause the display to display information comprising the second parameter.

16. The wearable health monitoring system of claim 15 wherein the first device is a cardioverter defibrillator class III medical device

17. The wearable health monitoring system of claim 15 wherein the first parameter includes an ECG signal.

18. The wearable health monitoring system of claim 15 wherein the second device is a motion sensing device.

19. The wearable health monitoring system of claim 15 wherein the second parameter includes a step count.

20. The wearable health monitoring system of claim 15 wherein the processor is remote from the first device and from the second device.
